(19) United States
(12) Patent Application Publication
(10) Pub. No.: US 2017/0189624 A1
(43) Pub. Date: Jul. 6, 2017

(54) Hypodermic Syringe Applicator Assist Device

(71) Applicant: Robert Byron Welborn, Granbury, TX (US)

(72) Inventor: Robert Byron Welborn, Granbury, TX (US)

(21) Appl. No.: 15/394,459

(22) Filed: Dec. 29, 2016

Related U.S. Application Data
(60) Provisional application No. 62/273,266, filed on Dec. 30, 2015.

(51) Int. Cl. A61M 5/32; A61M 5/315

(52) U.S. Cl. CPC A61M 5/3287; A61M 5/3202; A61M 5/31501; A61M 5/3158

(57) ABSTRACT

The present invention is directed to a device which facilitates injection of a medicament using a hypodermic syringe. An exemplary embodiment of the device includes an injection application for a hypodermic syringe comprising a main body, a cover, an axial force structure, and a rate controller. The main body includes a perimeter bounding and defining an enclosure about a primary axis and operable to mechanically grip and enclose a major section of the hypodermic syringe. The cover is opaque, extends from the main body, and is continuous and unitary with the main body. The axial force structure is disposed in the main body and is operable to provide axial travel force to the hypodermic syringe along the primary axis. A rate controller is in cooperation with the axial force, and is operable to control the rate of travel of the hypodermic syringe from the unimpeded axial force.

### BACKGROUND

Field of the Invention

This invention is directed to injection devices and particularly to an applicator for a hypodermic syringe.

Description of the Related Art

The requirement for self, or home-administered injections by non-medical personnel is becoming more common. Some of the reasons for this need are increased prescriptions of injectable medicaments including hormones and vitamins. For example, medicaments such as insulin have to be administered frequently and often at irregular times. Also, in many cases a person requiring an injectable medicament may be at less than full mental capacity to some degree, for example, in cases of asthma or reactions to such things as allergens, insect stings, or the like when an antidote, for example, atropine, epinephrine, or other medicament must be immediately injected. The recipient may not administer the medicament or be apprehensive or improperly inject the medicament due to anticipation or fear of pain. It would be advantageous to have a device which address these or other issues.

### SUMMARY

The present invention is directed to a device which facilitates injection of a medicament using a hypodermic syringe. An exemplary embodiment of the device includes an injection application for a hypodermic syringe comprising a main body, a cover, an axial force structure, and a rate controller. The main body includes a perimeter bounding and defining an enclosure about a primary axis and operable to mechanically grip and enclose a major section of the hypodermic syringe. The cover is opaque, extends from the main body, and is continuous and unitary with the main body. The axial force structure is disposed in the main body and is operable to provide axial travel force to the hypodermic syringe along the primary axis. A rate controller is in cooperation with the axial force, and is operable to control the rate of travel of the hypodermic syringe from the unimpeded axial force.

These and other features, aspects, and advantages of the invention will become better understood with reference to the following description, and accompanying drawings.

### BRIEF DESCRIPTION OF THE DRAWINGS

FIG. 1a depicts a side view of an embodiment of the current invention as it may exist in operation with the syringe in pre-application state;

FIG. 1b depicts a side view of an embodiment of the current invention as it may exist in operation with the syringe in post-application state;

FIG. 2a depicts an exploded side view of an embodiment of the current invention;

FIG. 2b depicts an exploded perspective side view of an embodiment of the current invention;

FIG. 3 depicts an exploded perspective side view of part of an embodiment of the current invention; and

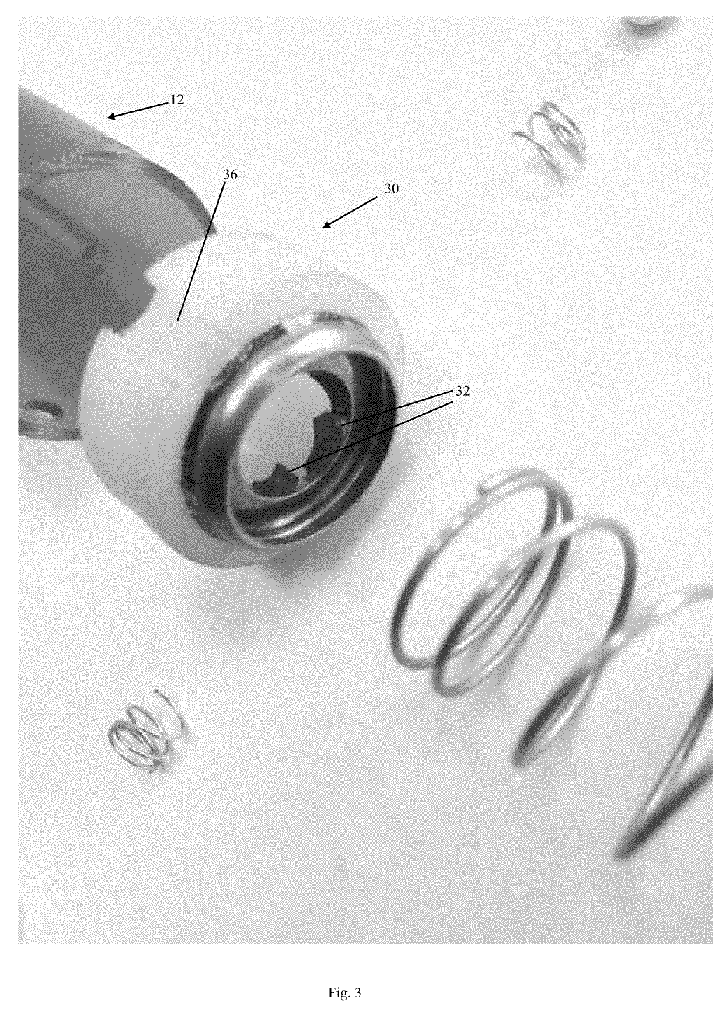

FIG. 4 depicts a top view of part of an embodiment of the current invention.

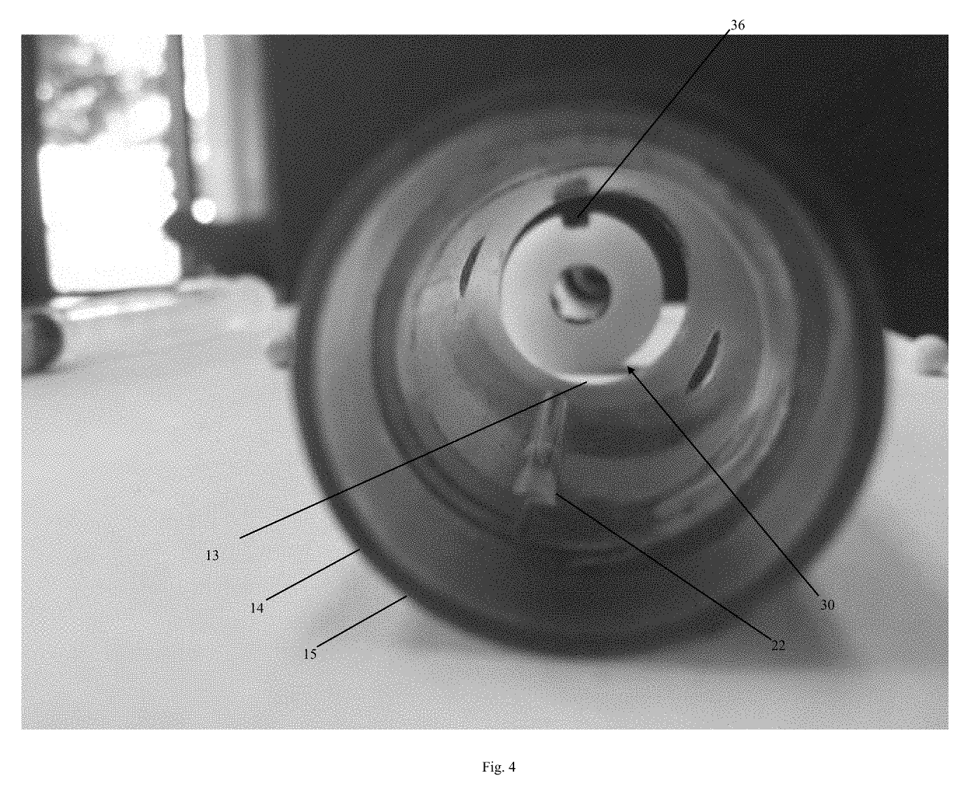

### DETAILED DESCRIPTION

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

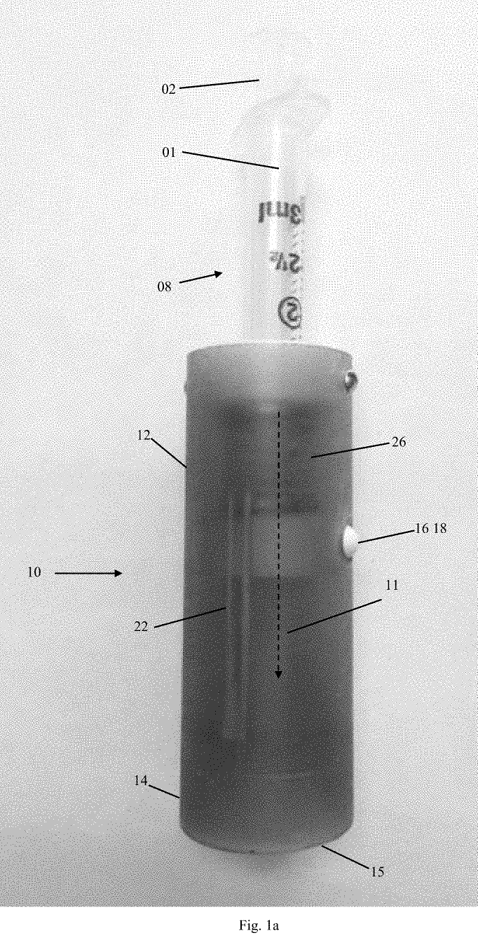

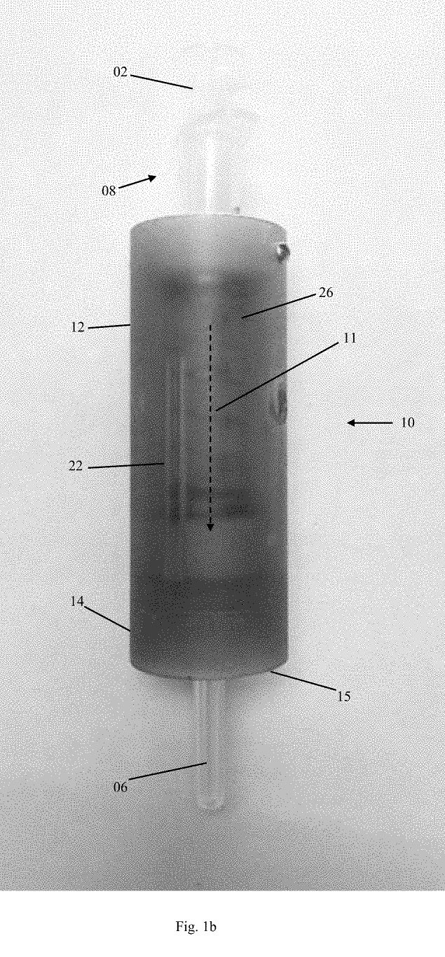

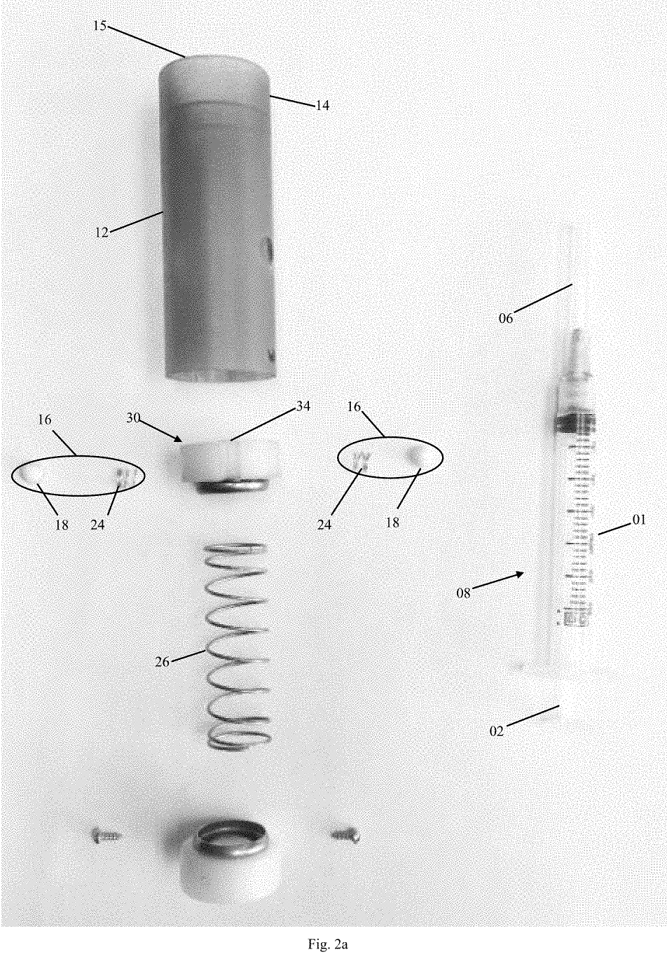

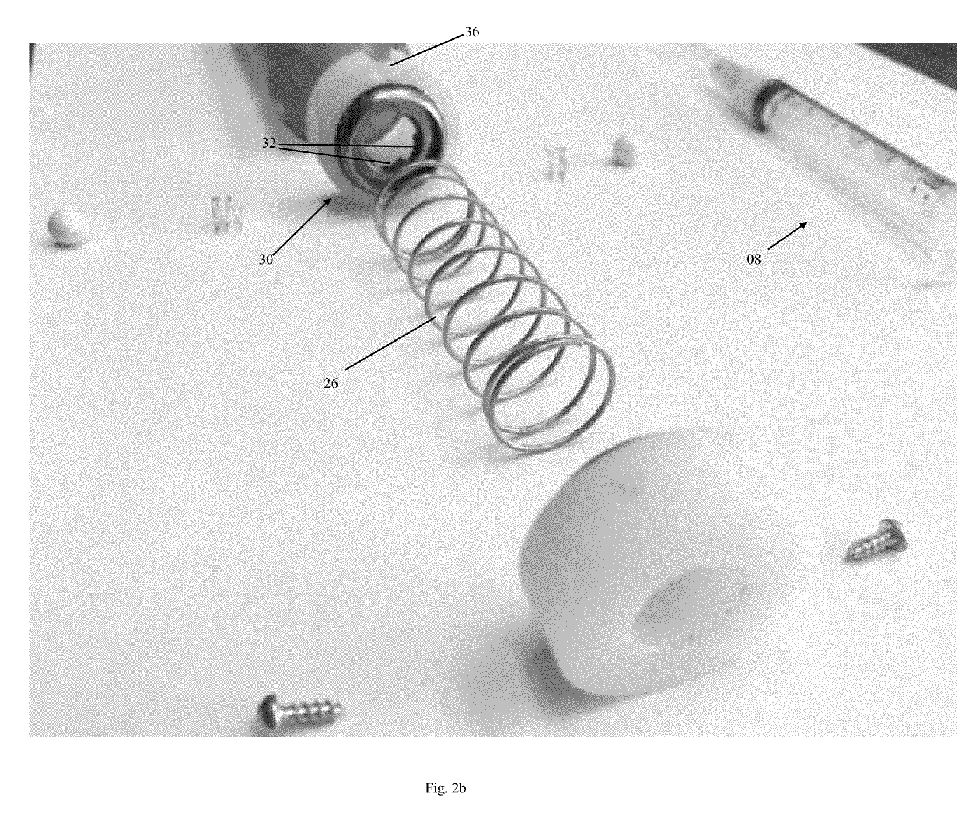

The present invention is directed to a device which facilitates injection of a medicament using a hypodermic syringe. FIGS. 1a and 1b illustrate embodiments of the device of the present invention, where FIG. 1a illustrates the device 10 with an engaged hypodermic syringe 08 prior to injection and FIG. 1b illustrates the device 10 with an engaged hypodermic syringe 08 after application of an injection. FIGS. 2a and 2b illustrate exploded views of embodiments of the device of the present invention. Depicted are a hypodermic syringe applicator assist device 10 and a hypodermic syringe 08.

A hypodermic syringe 08 is a simple pump 02 typically including a plunger that fits tightly in a barrel. The pump 02 can be pulled and pushed along inside the barrel, allowing the hypodermic syringe to take in or expel a liquid medicament 01 through an orifice at the open end of the barrel. The open end of the barrel is typically fitted with a needle 06, a nozzle, or tubing to help direct the flow into and out of the barrel. The needle 06 is used to penetrate the skin so that, by application of the pump 02, the medicament 01 can be directed from the barrel through the needle 06 in order to administer the injection.

In exemplary use, the hypodermic syringe applicator assist device 10 enables patients or caregivers who are directed to administer injections to inject the needle 06 of the hypodermic syringe 08 but self apply the pump 02 to deliver the medicament 01 without altering the hypodermic syringe 08. The depicted hypodermic syringe applicator assist device 10 includes a main body 12 and a cover 14.

The main body 12 is operable to mechanically grip and enclose a major section of the hypodermic syringe 08. In exemplary configuration, it encloses and holds the bottom section of the hypodermic syringe 08, leaving the pump 02 exposed for patient access. The main body 12 includes a perimeter bounding and defining an enclosure 13 about a primary axis 11. The main body 12 is compose of a rigid material such as durable PVC plastic. A suitable geometry of the main body 12 is generally cylindrical, although it may take other forms. The main body 12 is dimensioned to contain a major section of the hypodermic syringe 08. In exemplary configuration, its length is long enough to hold the body of the hypodermic syringe 08 while leaving the pump 02 exposed for patient access on one end and the needle 06 extending out of the opposing end, but still inside the cover 14 prior to injection of the needle 06.

A cover 14 extends from the main body 12 of the hypodermic syringe applicator assist device 10. In exemplary configuration, it is continuous and unitary with the main body 12, and composed of the same material of the main body 12. In further exemplary configuration, the cover 14 is opaque, blocking visual access to the enclosure 13 from the side. An exemplary length of the opaque cover 14 is enough to obscure the portion of the needle 06 exposed outside the body 12 in pre-application state while enabling the needle 06 to extend past the cover 14 in post-application state. An exemplary length of the opaque cover 14 is enough to obscure about ¼ inch portion of the needle 06 exposed outside the body 12 in pre-application state while enabling the needle 06 to extend about one inch past the cover 14 in post-application state. An exemplary width of the opaque cover is about 1 inch. The exemplary cover 14 further includes an edge 15 operable for resilient and sealed contact with the patient's skin. The depicted edge 15 is generally planar, although it is within the scope of the invention to employ other edge geometries.

The hypodermic syringe applicator assist device 10 also includes structure for providing axial force to a hypodermic syringe 08 engaged to the device 10. The axial force moves the hypodermic syringe 08 along the primary axis of the device 10. In the depicted configuration, a spring 26 is mounted in the axial travel path of the hypodermic syringe 08 such that it provides the axial force. The length, spring force, and other characteristics (for example, linear or non-linear) are selected. The deployed spring provides a configured axial force to drive the hypodermic syringe 08 through the body 12. The depicted configuration also includes an annular plunger 30 operable to receive the lower barrel end of the hypodermic syringe 08 such that the barrel abuts the plunger 30 while the needle 06 extends therethrough.

Other representative structures for axial force include springs, rubber bands, or others known in the art. For example, an elastic band, such as a rubber band can be employed. The elastic band is mounted with one end secured to the lower end of the main body 12. The upper end of the elastic band is secured to a movable section in the upper end of the main body 12, whereby an elastic force biased towards the lower end results.

The depicted configuration also includes a guide 22 on the interior surface of the main body 12 extending along the primary axis 11. A notch 36 is also included in the depicted plunger 30, operable to restrict axial motion of the plunger 30 when the notch 36 is not facing the guide 22 and permit axial motion of the plunger 30 when the notch is facing the guide 22. The illustrated plunger 30 also includes locking teeth 32 spaced about its interior, extending inwardly operable to engage the hypodermic syringe 08 and enable locking the syringe in varying rotational positions (˜¼ turn in the illustration). The depicted embodiment also includes a stop ring 34 proximate the bottom end of the main body 12 operable to stop travel of the plunger 30, in turn stopping the travel of a hypodermic syringe 08 contained therin.

A trigger 18 is cooperatively joined to the axial force structure in order to enable controlled activation of the spring force. The illustrated trigger is a button 18. In exemplary configuration, a safety lock 16 is incorporated into the trigger 18, operable to further control the condition(s) under which the axial force is delivered. The illustrated safety lock 16 includes a second button 18 on the opposing side of the main body 12. In order to activate the axial force, both buttons 18 must be depressed.

In exemplary configuration, a rate controller is also included and in cooperation with the axial force. The rate controller controls the rate at which the axial force is applied to the hypodermic syringe 08 so that the rate of injection of the needle can be controlled and steadied from the full snap force that would normally be delivered by an unimpeded axial force delivery. Exemplary rate controllers alter the unimpeded rate of the axial force and, in turn, the velocity at which the hypodermic syringe 08 travels. Exemplary rate controllers average the velocity or slope of the velocity curve. Representative rate controllers include frictional surface contacts 24 extending inwardly to contact the surface and opposing spring tension.

In one configuration, the buttons 18 are in cooperation contact with frictional surface contacts 24. In operation, when the buttons 18 are depressed, the springs 24 extend inwardly and contact the syringe barrel making frictional contact with the traveling hypodermic syringe 08. In an alternate configuration, a spring is mounted in the axial travel path of the hypodermic syringe 08 such that it provides an opposing force. The length, spring force, and other characteristics (for example, linear or non-linear) are selected. The deployed spring provides a configured counterforce to the primary axial force. In an alternate configuration, the plunger 30 is sized for sealable or near-sealable engagement to the interior wall of the main body 12 such that air flow between the interior of the cover region and the interior of the main body 12 is impeded. In use, a seal region is further presented by the plunger 30 seal, the cover 14 and the edge 15 engaged to the user skin, impeding the travel rate of the hypodermic syringe 08 contained therein. It is within the scope of the invention to combine one or more of the rate controllers.

Having described various configuration of the device, its use is described. In use, the patient retrieves the hypodermic syringe 08 and fills the syringe barrel with the medicament 01, leaving the pump 02 extending. The patient inserts the hypodermic syringe 08 into the enclosure 13 of the main body 12 and rotates it to lock it into position (˜¼ turn (or 90°) in the illustrated configuration). The pump 02 is exposed and the needle 06 is currently extending outside the main body 12 but still inside the opaque cover 14 as shown in FIG. 1a. The patient grabs the tab “ears” of the hypodermic syringe 08 to load spring 26 pressure. The opaque cover 14 blocks the needle 06 from patient view. The patient removes the needle 06 cover and places the opaque cover 14 against his skin so that the edge 15 is flush with his skin and the opaque cover 15 forms a seal. The patient simultaneously presses both buttons 18 as the safety lock 16 and the needle 06 is injected into the patient at the controlled rate due to the rate controller. The patient presses the pump 02 to deliver the medicament 01 and complete the injection.

Insofar as the description above and the accompanying drawing disclose any additional subject matter that is not within the scope of the single claim below, the inventions are not dedicated to the public and the right to file one or more applications to claim such additional inventions is reserved.

What is claimed is:

1. An injection application for a hypodermic syringe comprising:
a main body, a cover, an axial force structure, and a rate controller;
the main body including a perimeter bounding and defining an enclosure about a primary axis and operable to mechanically grip and enclose a major section of the hypodermic syringe;
the cover extends from the main body and continuous and unitary with the main body;
axial force structure disposed in the main body operable to provide axial travel force to the hypodermic syringe along the primary axis; and
a rate controller in cooperation with the axial force operable to control the rate of travel of the hypodermic syringe from the unimpeded axial force.
